(19) United States
(12) Patent Application Publication
(10) Pub. No.: US 2007/0140896 A1
(43) Pub. Date: Jun. 21, 2007

(54) Liquid toothbrush/dental appliance sanitizer

(75) Inventor: Melina Lavern Baggett, Sumrall, MS (US)

Correspondence Address: Melina Baggett, 101 Hickory Grove Church Road, Sumrall, MS, 39482, US

(73) Assignee: Melina Lavern Baggett, Sumrall, MS (US)

(21) Appl. No.: 11/305,549

(22) Filed: Dec. 15, 2005

(51) Int. Cl. A61L 2/18

(52) U.S. Cl. 422/28

(57) ABSTRACT

This invention relates to using a liquid form of sanitizer and disinfectant, particularly sodium hypochlorite or 2NaOH+Cl₂⇄NaCl 30 NaClO+H₂O in a 0.01-3% solution with H₂O and the addition of Sodium Chloride or NaCl to kill bacteria growing on toothbrushes and dental appliances caused by exposure to pathogenic microorganisms and microorganisms in the mouth. This invention will be made available to the public in an easy to use form for every day sanitizing. Other sanitizers on the market use steam, ozone, or Ultra-Violet light to disinfect toothbrushes and dental appliances. This will use a chemical mixture. Our independent clinical studies proved that the combination of chemicals suggested work to accomplish the goal.

### BACKGROUND OF INVENTION

The invention relates to using a liquid form of sanitizer and disinfectant to kill bacteria growing on toothbrushes and dental appliances caused by exposure to pathogenic microorganisms and microorganisms in the mouth.

Toothbrushes and dental appliances are generally exposed to air and pathogenic microorganisms in it. Microorganisms can transfer to a toothbrush or dental appliance located next to it and from toilet aerosol. Germs from the mouth build up on the toothbrush, and each time the toothbrush or dental appliance is placed in the mouth, those germs enter the body. Rinsing with water is not enough to kill these microorganisms.

When a toothbrush is brought into the laboratory and cultured, the amount of microorganisms that grow during this process are numerous.

### BRIEF SUMMARY OF INVENTION

With this invention, microorganisms are killed by soaking the toothbrush or dental appliance for a period of at least 10 minutes in the solution, followed by a rinse of the appliance.

The solution is designed to be harmless to humans because of the low concentration and be beneficial in killing germs such as those that cause Streptococcus pneumoniae and Escherichia coli.

### BRIEF DESCRIPTION OF THE SEVERAL VIEWS OF THE DRAWING

“The patent or application file contains at least one drawing executed in color. Copies of this patent or patent application publication with color drawing(s) will be provided by the Office upon request and payment of the necessary fee.”

FIG. 1—Toothbrush after four weeks of normal use. Blood Agar Plate at room temperature shows heavy growth of many colonies after 48 hours.

FIG. 2—The same toothbrush which had been soaking in our solution was used. Blood Agar Plate at room temperature shows no growth after 48 hours. The five colonies that are on the plate were not in the area that was set up.

FIG. 3—Used a sterile swab to pick up many colonies of E. coli and Strep. pneumo. Put swab in our chemical solution for then minutes and set up on Blood Agar Plate at room temperature. No growth was shown at 48 hours.

FIG. 4—Additional picture of FIG. 3 with the addition of a partial view of a Blood Agar Plate growing the E. coli and Strep. pneumo. at room temperature after 48 hours of growth. Notice the many colonies developing.

### DETAILED DESCRIPTION OF THE INVENTION

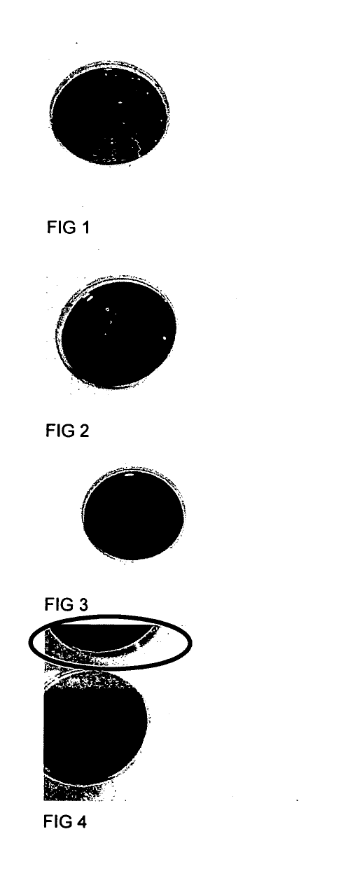

The sanitizing and disinfecting of toothbrushes and dental appliances is useful to help eliminate the spread of microorganisms that occur naturally or through aerolization. This invention will be used to create a kit for ordinary people to use to disinfect their toothbrushes/dental appliances in their homes using the chemicals provided already mixed. FIG. 1 shows the growth of bacteria from an ordinary toothbrush after only four weeks of normal use. FIG. 2 shows the growth of bacteria (or lack thereof) after soaking in the chemical mixture.

Other sanitizers on the market use steam, ozone, or Ultra-Violet light to disinfect toothbrushes and dental appliances. This will use a chemical mixture.

The chemical mixture intended is: Sodium hypochlorite or 2NaOH+Cl₂ [symbol] NaCl+NaClO+H₂O in a 0.01-3% solution with H₂O and the addition of Sodium Chloride or NaCl.

A soaking period of at least 10 minutes is necessary for this elimination of microorganisms. FIG. 3 shows a set up of the Blood Agar Plate after a sterile swab was used to pick up colonies of Streptococcus pneumoniae and Escherichia coli. The swab was soaked for 10 minutes in the chemical mixture and no growth occurred after 48 hours at room temperature. FIG. 4 shows the same picture in FIG. 3 with the addition of a second Blood Agar Plate in the top left. This plate was evidence of an additional swab that had been used to pick up Streptococcus pneumoniae and Escherichia coli. There are many colonies growing in this plate.

Toothbrush or dental appliance will need to be rinsed before applying toothpaste or entering the mouth.

The chemical mixture will be marketed pre-mixed either in a container that is inserted in a specially designed toothbrush or dental appliance holder or in a bottle that will allow the consumer to pour the mixture into a specially designed toothbrush or dental appliance holder.

The chemical mixture will need to be replaced weekly.

1. Soaking a toothbrush or other dental appliance in a chemical solution of sodium hypochlorite or 2NaOH+Cl₂⇄NaCl+NaClO+H₂O in a 0.01-3% solution with H₂O and the addition of Sodium Chloride or NaCl is effective in killing microorganisms that grow on toothbrushes and other dental appliances that would otherwise been able to incubate.
